(19) United States
(12) Patent Application Publication
(10) Pub. No.: US 2025/0161634 A1
(43) Pub. Date: May 22, 2025

(54) CATHETER FIXATION DEVICE FOR DIFFERENT SIZE CATHETERS

(71) Applicant: Ramot Biomedical Engineering Ltd., Maas (IL)

(72) Inventors: Yair Ramot, Maas (IL); Roi Ramot, Rosh Haayin (IL); Ronny Barak, Tel Aviv (IL)

(73) Assignee: Ramot Biomedical Engineering Ltd., Maas (IL)

(21) Appl. No.: 18/955,092

(22) Filed: Nov. 21, 2024

Related U.S. Application Data
(60) Provisional application No. 63/601,767, filed on Nov. 22, 2023.

(51) Int. Cl. A61M 25/02

(52) U.S. Cl. CPC A61M 25/02; A61M 2025/0253

(57) ABSTRACT

A catheter fixation device includes a body including a floor, an entry port and an exit port. The body receives therein a catheter that passes through the entry and exit ports. The device includes an anti-pull-out lock mechanism that includes outer restraining members, each of which includes a vertical portion from which radially inwards extends a horizontal portion, and inner restraining members, at least one of includes an upper slanted abutment and a lower slanted abutment.

### FIELD OF THE INVENTION

The present invention relates generally to devices for supporting and fixing a catheter extending from an opening in a subject, for example a supply catheter or a drain catheter.

### BACKGROUND OF THE INVENTION

Many medical procedures conducted on a patient require the patient to be provided with a catheter, tube, line and the like (the terms being used interchangeably throughout the disclosure and claims). The catheter typically is introduced through an incision in the skin of the patient (or other opening, including a natural orifice). Such catheters are typically used to deliver fluids to the patient or to remove fluids from the patient.

In the prior art, catheters are secured to the patient after insertion and placement, such as by means of sutures, for example, as part of or in addition to the procedure for closing the incision. Alternatively, or in addition thereto, the drain line is secured to the skin using adhesive tape or surgical staples. However, clinical follow up on catheter placement and fixation reports a high rate of complications including leakage, obstruction, dislodgement, migration, malposition, and infection.

Thus an improved catheter fixation device is clearly needed.

### SUMMARY OF THE INVENTION

The present invention seeks to provide an improved catheter fixation device, as is described more in detail hereinbelow. The catheter fixation device of the invention provides improvements to the catheter fixation device described in PCT patent application WO 2021/064623 to the same applicant.

The catheter fixation device structure includes, without limitation, an anti-pull-out lock mechanism and enables controllable catheter maneuverability.

The catheter fixation device may be attached to the skin of the patient with an adhesive pad, which is simple to apply and assemble. The device may have a cover that enables easy monitoring, cleaning and maintenance of the device.

There is thus provided in accordance with a non-limiting embodiment of the present invention a catheter fixation device that includes a body including a floor, an entry port and an exit port, the body configured to received therein a catheter that passes through the entry port and the exit port, and an anti-pull-out lock mechanism including outer restraining members, each of which includes a vertical portion from which radially inwards extends a horizontal portion, and inner restraining members, at least one of includes an upper slanted abutment and a lower slanted abutment.

In accordance with a non-limiting embodiment of the present invention the exit port is an aperture formed in the body, and which is positioned near the floor, off-center from a center of the body.

In accordance with a non-limiting embodiment of the present invention the lower slanted abutment is arcuate.

In accordance with a non-limiting embodiment of the present invention the body includes a limiter element at the entry port, configured to restrain upper movement of a catheter placed in the body.

In accordance with a non-limiting embodiment of the present invention the limiter element includes markings to mark positions of different size catheters.

In accordance with a non-limiting embodiment of the present invention the body includes an attachment surface for attaching the body to a patient.

### BRIEF DESCRIPTION OF THE DRAWINGS

The present invention will be understood and appreciated more fully from the following detailed description, taken in conjunction with the drawing in which:

FIG. 1 is a simplified top view illustration of a catheter fixation device, constructed and operative in accordance with a non-limiting embodiment of the present invention.

FIG. 2 is a simplified side-view illustration of the catheter fixation device as viewed by arrow 2 in FIG. 1.

FIG. 3 is a simplified front-view illustration of the catheter fixation device as viewed by arrow 3 in FIG. 1.

FIG. 4 is a sectional illustration of the catheter fixation device, viewed as in FIG. 2.

### DETAILED DESCRIPTION OF EMBODIMENTS

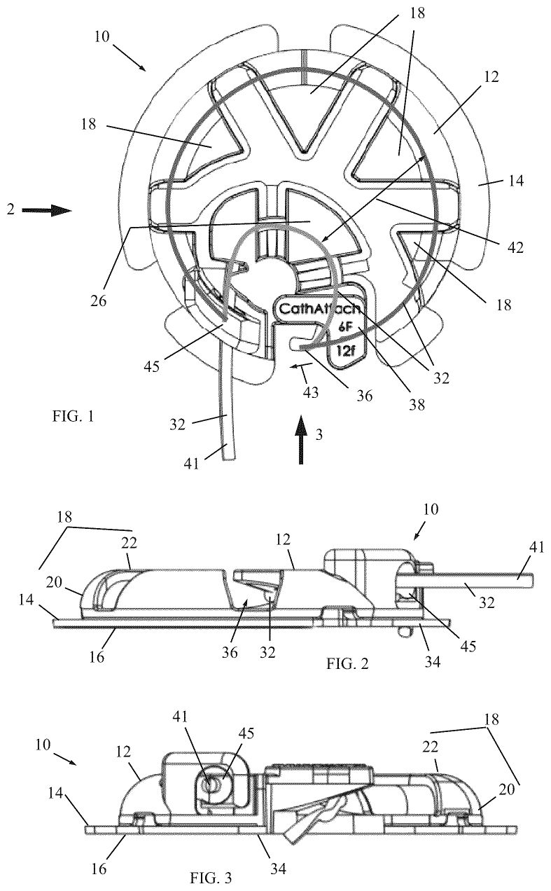

Reference is now made to FIG. 1-3, which illustrate a catheter fixation device 10, constructed and operative in accordance with a non-limiting embodiment of the present invention.

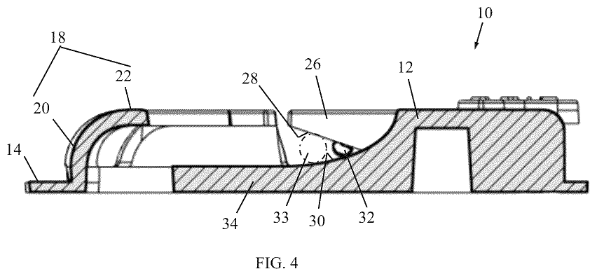

The catheter fixation device 10 includes a main body 12, which may be, but not necessarily, circular or elliptical. Body 12 may include an outer rim 14 which has an attachment surface 16 (in the sense of the drawings, the bottom surface, shown in FIGS. 2 and 3), such as but not limited to, an adhesive surface.

The catheter fixation device 10 includes an anti-pull-out lock mechanism and enables controllable catheter maneuverability. Without limitation, the anti-pull-out lock mechanism includes outer restraining members 18, each of which may include a vertical portion 20 from which radially inwards extends a horizontal portion 22 (“vertical” and “horizontal” in the sense of the drawings). In the illustrated embodiment, the horizontal portion 22 may have a triangular shape but other shapes are also contemplated in the scope of the invention, such as polygonal or irregular shapes. A junction 24 (FIG. 4) between vertical portion 20 and horizontal portion 22 may be rounded.

The anti-pull-out lock mechanism may also include inner restraining members 26, each of which or at least one of which may include an upper slanted abutment 28 (FIG. 4) and a lower slanted abutment 30 (“upper” and “lower” in the sense of the drawings). The lower slanted abutment 30 may be arcuate, as seen in FIG. 4.

As seen in FIG. 4, because the upper slanted abutment 28 is slanted, it provides several locations for catheters of different diameters to abut against. For example, a 6 Fr (2 mm OD) catheter 32 can abut against a more radially inner portion of upper slanted abutment 28 near lower slanted abutment 30. The upper movement of catheter 32 is restrained by upper slanted abutment 28, and the lower movement of catheter 32 is restrained by lower slanted abutment 30. A 12 Fr (4 mm OD) catheter 33 (shown in broken lines in FIG. 4) can abut against a more radially outer portion of upper slanted abutment 28. The upper movement of catheter 33 is restrained by upper slanted abutment 28, and the lower movement of catheter 33 is restrained by a lower floor 34 of body 12.

The catheter, such as catheter 32 as shown in FIG. 1, may enter device 10 at an entry port 36, which is located at a circumferential gap formed in body 12. The entry port 36 is where the catheter exits the patient's body and enters the device. The portion of the catheter that enters the body through the skin is bent downwards (in the sense of the drawings).

After entry into the device, the catheter 32 may wind or wrap around an arcuate channel 42 formed between outer restraining members 18 and inner restraining members 26, and finally a portion 41 of the catheter 32 exits through an exit port 45 in body 12. Exit port 45 may be an aperture formed in body 12, which is positioned near the floor 34 of body 12, off-center from the center of body 12. Thus, the coiled portion of the catheter 32 is constrained to move radially between outer restraining members 18 and inner restraining members 26 during movement of the proximal portion of the catheter while the portion of the catheter 32 at the entry port 36 remains substantially in place.

When pulled in the proximal direction (shown by arrow 43 in FIG. 1), the coiled portion of the catheter can abut against the inner restraining members 26; when not pulled in the proximal direction, the coiled portion of the catheter can abut against the outer restraining members 18. The floor 34 prevents downward (in the sense of the drawings) pull-out of the catheter.

Body 12 may include a limiter element 38 at the entry port 36, which restrains the upper movement of the catheter. In accordance with an embodiment of the invention, limiter element 38 may include markings 40 to mark the position of different size catheters, such as but not limited to, the 6 Fr catheter 32 and the 12 Fr catheter 33.

As known in the art, the end 41 of the catheter may be coupled to a fluid component, such as but not limited to, a luer lock, pinch lock and others (not shown). The other end of the catheter that enters the patient's body may include, without limitation, a needle or sharp end or non-sharp end (not shown).

The catheter fixation device keeps the catheter safely in place in the skin of the patient, while permitting movement of the portion of the catheter that has exited the device. The portion of the catheter near entry into the patient is prevented from moving.

The catheter fixation device 10 may be made of injection-molded plastic or any medically safe material, with different hardness and flexibility, as required.

The device 10 may have a cover (not shown), which enables easy monitoring, cleaning and maintenance of the device. The cover may block view of the catheter so that it is more pleasant for most users or can be transparent to enable monitoring the catheter position and hygiene. The cover may help keep the catheter in place so it does not pull out.

What is claimed is:

1. A catheter fixation device comprising:
a body comprising a floor, an entry port and an exit port, said body configured to received therein a catheter that passes through said entry port and said exit port; and
an anti-pull-out lock mechanism comprising outer restraining members, each of which comprises a vertical portion from which radially inwards extends a horizontal portion, and inner restraining members, at least one of comprises an upper slanted abutment and a lower slanted abutment.

2. The catheter fixation device according to claim 1, wherein said exit port is an aperture formed in said body, and which is positioned near said floor, off-center from a center of said body.

3. The catheter fixation device according to claim 1, wherein said lower slanted abutment is arcuate.

4. The catheter fixation device according to claim 1, wherein said body comprises a limiter element at said entry port, configured to restrain upper movement of a catheter placed in said body.

5. The catheter fixation device according to claim 4, wherein said limiter element comprises markings to mark positions of different size catheters.

6. The catheter fixation device according to claim 1, further comprising a catheter having a proximal portion and a distal portion that passes through said entry port and said exit port.

7. The catheter fixation device according to claim 1, wherein said body comprises an attachment surface for attaching said body to a patient.
